(19) United States
(12) Patent Application Publication
(10) Pub. No.: US 2013/0172781 A1
(43) Pub. Date: Jul. 4, 2013

(54) pH INDICATOR MODULE FOR GASTRIC TUBES

(71) Applicant: Dale Medical Products, Inc., Plainville, MA (US)

(72) Inventor: Ronald D. Russo, Naples, FL (US)

(73) Assignee: Dale Medical Products, Inc., Plainville, MA (US)

(21) Appl. No.: 13/657,247

(22) Filed: Oct. 22, 2012

Related U.S. Application Data
(60) Provisional application No. 61/627,951, filed on Oct. 21, 2011.
(60) Provisional application No. 61/686,275, filed on Apr. 3, 2012.

(51) Int. Cl. A61B 5/145

(52) U.S. Cl. CPC A61B 5/14507
USPC 600/584

(57) ABSTRACT

A pH indicator module is configured to attach on a proximal end to a suction mechanism and on a distal end to a proximal end of an aspiration tube. The pH indicator module comprises a barrel connector configured to attach to an aspiration tube, the barrel connector forming an inner passageway, the passageway being located in a direct flow path of the aspirated fluid when the pH indicator module is used. The barrel connector is configured to receive a pH indicator material placed in the passageway, the pH indicator material being configured to come into contact with a direct flow of the aspirated fluid drawn into the passageway to determine a pH value of the aspirated fluid. The pH indicator module also comprises a cap portion configured to attach on a proximal cap end to the suction mechanism and to attach on a distal cap end to the barrel connector.

### PRIORITY

This application claims priority under 35 U.S.C. §119 to U.S. Provisional Patent Application No. 61/627,951, filed on Oct. 21, 2011; and U.S. Provisional Patent Application No. 61/686,275, filed on Apr. 3, 2012, both of which are herein incorporated by reference.

### FIELD

The present disclosure relates to pH indicators, and more specifically, to pH indicators for aspirated fluids.

### BACKGROUND

Nasogastric medical tubes are typically placed to decompress the stomach or to deliver liquid enteral nutrition formula into the stomach. During proper intubation, the distal tip of the tube enters the esophagus and is pushed down into the stomach. Due to erroneous intubation, on the other hand, the distal tip of the tube may veer off into the airway bronchus. In such a case, liquids or enteral formula may be sent into the lungs, causing catastrophic consequences for the patient. As such, before the administration of any liquid medication or enteral formula through the nasogastric tube, most hospital or critical care protocols require verification that the distal end of the tube is actually in the stomach.

A few methods have previously been used to verify placement of the nasogastric tube into the stomach. These methods include x-ray verification, stethoscope, auscultation, and pH testing of syringe aspirated stomach fluid. The x-ray verification uses an x-ray of the torso of the patient to determine the location of the already inserted tube. While the x-ray method can be accurate, it may not be feasible, economic, or harmless. Bedside x-ray is not always readily available. Moreover, exposing the patient to the x-ray radiation can be harmful.

A more feasible method for confirming gastric placement of nasogastric tubes endorsed by the American Association of Critical Care Nurses is the pH testing of aspirated stomach fluid. Stomach fluid, or gastric fluid, is generally agreed to have a pH of 4.5 or less, which falls in the acidic range. The blue litmus paper is a convenient tool for detecting this pH, because, upon contact with a liquid with a pH of 4.5 or less, the paper changes colors from blue to red.

The standard pH testing procedure, however, requires considerable time, work and effort to gather all the necessary supplies. Moreover, the pH testing procedure is often messy and potentially exposes the user or clinician to potentially infectious aspirated contents. The procedure requires the clinician to acquire litmus pH indicator paper, a medicine cup, and a 60 cc piston syringe having a catheter tip. The clinical then has to aspirate stomach fluid from the tube using a 60 cc piston-syringe, transfer this fluid to the medicine cup, and then manually dip the litmus paper into the cup and accordingly determining the pH. An acidic pH usually indicates that the distal tip of the tube is properly placed in the stomach. To reduce the steps of the manual procedure, US patent application publication 2011/0077495 by Gilbert discloses a housing that is inserted at the end of a nasogastric tube. The housing includes a detection indicator 5504 that changes between two visual indications upon contact with a fluid.

An object of the invention is to provide an efficient, cost effective, and simple to use apparatus for determining the pH of the aspirated gastric fluid.

### SUMMARY

In various embodiments, a pH indicator module configured to attach on a proximal module end to a suction mechanism and on a distal module end to a proximal tube end of an aspiration tube, and configured to be used during aspirating an aspirated fluid drawn by the suction mechanism from a location of a distal tube end of the aspiration tube, the pH indicator module comprising: a barrel connector configured to attach to the aspiration tube, wherein the barrel connector forms an inner passageway and wherein the passageway is located in a direct flow path of the aspirated fluid when the pH indicator module is used, and wherein the barrel connector is configured to receive a pH indicator material placed in the passageway, wherein the pH indicator material is configured to come into contact with a direct flow of the aspirated fluid drawn into the passageway to determine a pH value of the aspirated fluid; and a cap portion configured to attach on a proximal cap end to the suction mechanism and to attach on a distal cap end to the barrel connector.

In some embodiments, the pH indicator material is a pH indicating strip of material. In some embodiments, the pH indicator material is shaped in a rectangle and is folded in a V shape cross section before being placed in the passageway. In some embodiments, the pH indicating strip is fixed inside the passageway, at least in part, by contacting an inner surface of the passageway. In some embodiments, the barrel connector is made of a transparent material, and wherein the pH indicator material is visible from a viewing angle greater than or equal to 180° around the pH indicator module. In some embodiments, the barrel connector is sized such that during the aspirating less than 0.5 ml of the aspirated fluid is required to enter the pH indicator module for the aspirated fluid to come into contact with the pH indicator material.

In some embodiments, a slot is formed in the passageway and wherein the pH indicating strip of material is inserted in the slot and held in place, at least in part, by contacting internal walls of the slot. In some embodiments, the pH indicator module further comprises a label including a label color that matches an indicator color of the pH indicator material when during the aspirating the pH indicator material comes into contact with the aspirated fluid and when the aspirated fluid has a pH value in a specific range. In some embodiments, the cap portion includes at the proximal cap end a bushing section configured to attach to the suction mechanism and further includes at the distal cap end a cap section configured to attach to the barrel connector. In some embodiments, the pH indicator material is configured to determine that the pH value falls into one of more than two pH value ranges. In some embodiments, the proximal cap end is configured to be connected to a syringe.

In some embodiments, the pH value is used to determine whether a distal tube end of a gastric tube has been properly placed within a patient's stomach. In some embodiments, the pH indicator material is placed in a center of the passageway. In some embodiments, the passageway is cylindrical. In some embodiments, the pH indicator module further comprises the pH indicator material, wherein the pH indicator material is inserted and held in place in the passageway, such that the pH indicator material is configured to come into contact with the direct flow of the aspirated fluid drawn into the passageway. In some embodiments, the proximal cap end includes a luer fitting configured to be connected to a luer syringe.

In various embodiments, a method is disclosed of using a pH indicator module to measure a pH value of an aspirated fluid, wherein the pH indicator module is configured to attach on a proximal module end to a suction mechanism and on a distal module end to a proximal tube end of an aspiration tube, and to be used during aspirating the aspirated fluid drawn by the suction mechanism from a location of a distal tube end of the aspiration tube. The method comprises providing a barrel connector included in the pH indicator module, the barrel connector configured to attach to the aspiration tube, wherein the barrel connector forms an inner passageway, and wherein the passageway is located in a direct flow path of the aspirated fluid when the pH indicator module is used; providing a pH indicator material inserted in the passageway, wherein the pH indicator material is configured to come into contact with a direct flow of the aspirated fluid drawn into the passageway to determine a pH value of the aspirated fluid; and providing a cap portion included in the pH indicator module, wherein the cap portion is configured to attach to the suction mechanism on a proximal cap end and to attach to a proximal barrel end of the barrel connector on a distal cap end; attaching a distal barrel end of the barrel connector to the aspiration tube; drawing a fluid into the passageway using the suction mechanism; and observing a color of the pH indicator material after contacting the fluid.

In some embodiments, the pH indicator material is a pH indicating strip of material, and the pH value is used to determine whether a distal tube end of a gastric tube has been properly placed within a patient's stomach.

In some embodiments, the method further comprises providing a label including a label color that matches an indicator color of the pH indicator material when during the aspirating the pH indicator material comes into contact with the aspirated fluid and when the aspirated fluid has a pH value in a specific range. In some embodiments, the cap portion includes at the proximal cap end a bushing section configured to attach to the suction mechanism and further includes at the distal cap end a cap section configured to attach to the barrel connector.

### BRIEF DESCRIPTION OF THE DRAWINGS

The drawings are not necessarily to scale. Instead, emphasis is generally placed upon illustrating the principles of the inventions described herein. It is to be understood that the following detailed description is exemplary and explanatory only and is not restrictive of any invention, as claimed. The accompanying drawings, which are incorporated in and constitute a part of this specification, illustrate several embodiments consistent with the inventions and together with the description, serve to explain the principles of the inventions. In the drawings:

FIGS. 1A-1F depict different views of a pH indicator module and its components according to various embodiments.

FIGS. 2A and 2B show a fluid aspiration system, which utilizes a pH indicator module according to various embodiments.

FIG. 3 shows a system in which pH indictor module attaches to an ACE Connector® 300, according to an embodiment.

FIG. 4 shows a system in which a pH indictor module is used with an aspiration suction source other than a syringe, in accordance with some embodiments.

FIGS. 5A-5C depict a pH indicator and its components according to an embodiment.

FIGS. 6A-6C depict a pH indicator and its components according to another embodiment.

FIGS. 7A and 7B depict functions of a busing according to an embodiment.

FIG. 8 depicts a pH indicator package according to some embodiments.

### DETAILED DESCRIPTION

The following detailed description refers to the accompanying drawings. Wherever possible, the same reference numbers may be used in the drawings and the following description to refer to the same or similar parts. Also, similarly-named elements may perform similar functions and may be similarly designed. Numerous details are set forth to provide an understanding of the embodiments described herein. In some cases, the embodiments may be practiced without these details. In other instances, well-known techniques and/or components may not be described in detail to avoid obscuring described embodiments. While several exemplary embodiments and features are described herein, modifications, adaptations, and other implementations are possible, without departing from the spirit and scope of the invention. Accordingly, the following detailed description does not limit the invention. Instead, the proper scope of the invention is defined by the appended claims.

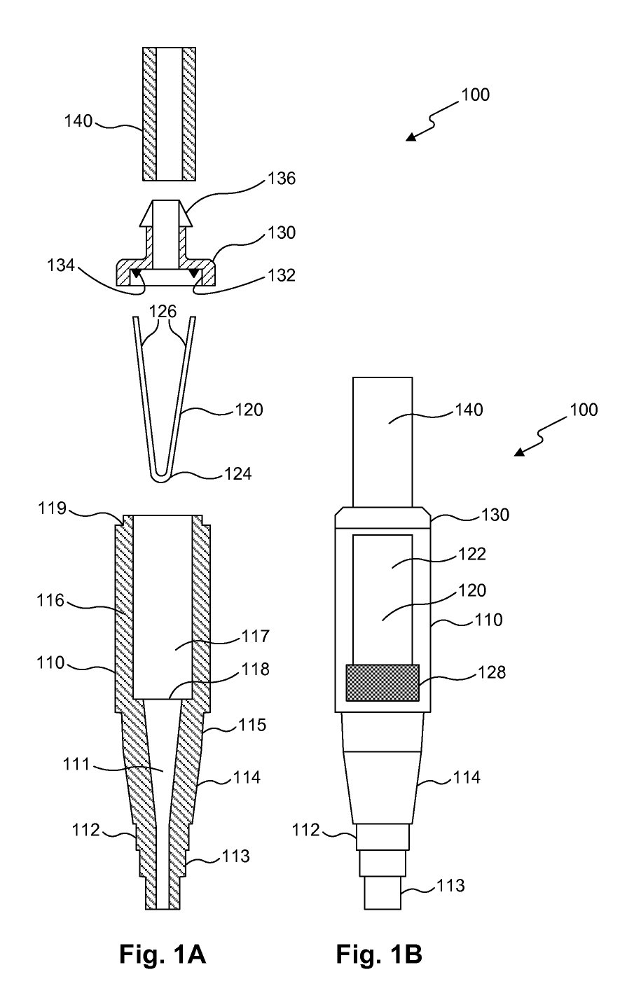

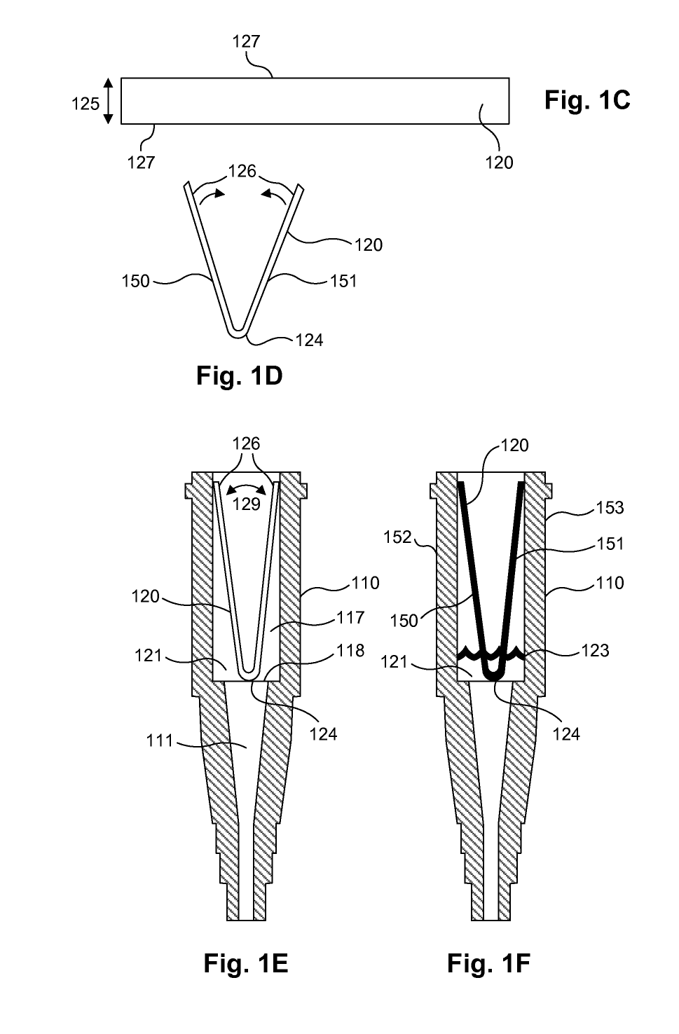

FIG. 1A is a side view of an assembled pH indicator module 100, and FIG. 1B is a frontal view of a disassembled pH indicator module 100 according to some embodiments. pH indicator module 100 includes a barrel connector 110, an indicator strip 120, a cap 130, and a bushing 140. In some embodiments, pH indicator module 100 also includes a label 128. Bushing 140, forming a proximal end of pH indicator module 100, is configured to connect to a syringe used for aspiration. Barrel connector 110, forming a distal end of pH indicator module 100, is configured to connect to an aspiration tube through a nozzle type tip 113, and to receive aspirated fluid. In various embodiments, for ease of reference, a first end of various parts is termed a proximal end and a second end of various parts is termed a distal end. In some embodiments, a proximal end is an end that will be located closer to the clinician when the part will be in use and a distal end is an end that will be located closer to the patient or placed inside the patient when the part will be in use.

Indicator strip 120, located inside barrel connector 110, comes into contact with the aspirated fluid and changes color if the pH of the aspirated fluid is in specific pH range. Label 128 shows one or more colors as guides for comparison with the color of the indicator strip 120 and for determining the pH of the aspirated fluid. In some embodiments, cap 130 and bushing 140 are molded as a once piece cap portion, which connects to a syringe on its proximal end and to barrel connector 110 on its distal end.

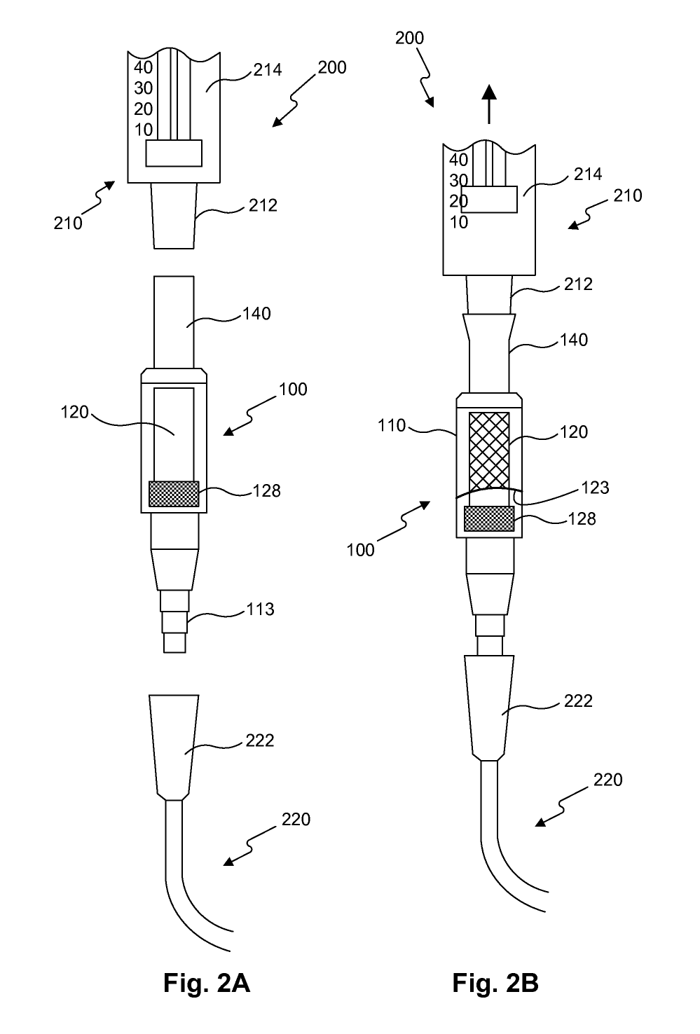

FIGS. 2A and 2B show a fluid aspiration system 200 which utilizes pH indicator module 100, according to various embodiments. Fluid aspiration system 200 includes a piston syringe 210, pH indicator module 100, and an aspiration tube 220. In the embodiment shown in FIGS. 2A and 2B, syringe 210 is a catheter tip syringe, e.g., a 50 cc syringe, which includes a plunger 214 and ends at a syringe tip 212. Aspiration tube 220 includes at its proximal end a tube terminal 222. pH indicator module 100 includes at its proximal end bushing 140 configured to receive syringe tip 212. pH indicator module 100 further includes, at its distal end, nozzle type tip 113 configured to be inserted into tube terminal 222.

To assemble fluid aspiration system 200, piston syringe 210 is attached to the proximal end of pH indicator module 100 by inserting tip 212 of piston syringe 210 into bushing 140 of pH indicator module 100. Further, nozzle type tip 113 of barrel connector 110, at the distal end of pH indicator module 100, is inserted into tube terminal 222 of aspiration tube 220. In some embodiments, aspiration tube 220 is a stomach tube, a distal end of which is configured to enter a patient's stomach. Using plunger 214, a user can aspirate fluid from the location of the distal end of aspiration tube 220 into barrel connector 110.

FIG. 1B is an exploded disassembled view of a pH indicator module 100, showing its parts according to some embodiments. In various embodiments, barrel connector 110 is a rigid one-piece injection molded portion made from a clear plastic such as polycarbonate. In the embodiment shown in FIG. 1B, barrel connector 110 is divided into multiple sub-sections, including a cap connector portion 119 at a proximal end, a barrel portion 116 in the middle, and a tube connector portion 112 at a distal end. In the embodiment shown, tube connector portion 112 includes a small stepped nozzle type tip 113 at the distal end, followed respectively by an enlarged tapered portion 114 and a flared portion 115.

In various embodiments, tube connector portion 112 is configured to enable connection to a wide variety of tubes or catheters, for example, stomach tubes such as nasogastric tubes and gastrostomy tubes. In some embodiments, enlarged tapered portion 114 matches the tapered angle of most 60 cc catheter tip piston syringes. In some embodiment, enlarged tapered portion 114 thereby enables a wedge lock angled leakproof engagement for the pH indicator module 100 to Dale® Medical Products, Inc. ACE Connector enteral connector syringe port seal. In some embodiments, flared portion 115 enables connection to the funnel end of balloon replacement gastrostomy tubes. In some embodiments, tube connector portion 112 is about 1¼ inches long with a beginning tip outside diameter of 0.185 inches. These dimensions provide a safety measure, preventing tube connector portion 112 from connecting to any female I.V. luer. In some embodiments, barrel connector 110 is about 2¼ inches long with an outside diameter of about 0.430 inches.

In some embodiments, the total length of indicator module 100 is about 3½ inches. Such a size provides a compact assembly that is about the size of a disposable 5 cc syringe.
In various embodiments, tube connector portion 112 and barrel portion 116 form at their interiors two interconnected hollow channels as passageways for the aspirated fluid. In particular, tube connector portion 112 forms connector passageway 111 and barrel portion 116 forms barrel passageway 117. Connector passageway 111 and barrel passageway 117 are configured to be in fluid communication when pH indicator 100 is used. The transition area between connector passageway 111 and barrel passageway 117, that is, where they join, forms an internal stop ledge element 118. In various embodiments, stop ledge 118 is formed by a difference in diameters of connector passageway 111 and barrel passageway 117 at the transition area. In some embodiments, at the transition area, connector passageway 111 has a circular opening of about 0.175 inches in diameter with and barrel passageway has a circular opening of about 0.285 inches in diameter. The differences in these diameters provide the circular flat circular stop ledge element 118.

In some embodiments, cap 130 is made by injection molding from rigid polycarbonate clear transparent plastic. In the embodiment shown in FIG. 1B, cap 130 forms internal sidewalls 132 configured to generate a slight press fit with cap connector portion 119 of barrel connector 110. Cap 130 also has a molded in sonic energy director ring 134. In some embodiments, energy director ring 134 is formed in a V shape. In some embodiments, the V shape has a height of about 0.015 inches. In some embodiments, ring 134 is melted during sonic welding to form a glueless permanent fusion with connector portion 119.

In various embodiments, cap 130 is a plastic injection molded component incorporating a molded in barb portion 136. Barb portion 136 enables a press on engagement with flexible molded or extruded bushing 140, permitting attachment to various standard catheter tip or generic tip syringe. In some embodiments, cap 130 is molded with a terminal end having a uniform dimensioned female luer portion instead of the barb portion 136. In some embodiments, female luer portion is molded in conformance with ISO/ANSI 1986 standard for luer fittings. In some embodiments, use of a female luer configuration enables a direct attachment of cap 130 with a variety of male luers or male luer lock aspiration syringes, thus eliminating the need for bushing 140. In some embodiments, module 100 is pre-connected and assembled with a luer or luer lock syringe, providing a ready to use pH indicator.

In some embodiments, bushing 140 has a cylindrical shape and is made of flexible PVC or flexible synthetic rubber. In the embodiment shown in FIG. 1B, bushing 140 is configured to fit over barb 136. In various embodiments, bushing 140 is either molded or extruded. In some embodiments, bushing 140 has an inner diameter of about 0.215 inches and a wall thickness of about 0.080 inches. In some embodiments, bushing 140 is of about 60 shore A flexible hardness. In various embodiments, bushing 140 enables connection to a variety of known catheter tip or enteric tip syringes.

In various embodiments, indicator strip 120 is configured to indicate, by its color, a pH of the aspirated fluid that contacts the strip. In some embodiments, strip 120 is a blue litmus paper, which changes color based on a pH of the fluid. In various embodiments, strip 120 is a coated paper pH indicator strip or a plastic strip. In various embodiments, strip 120 can provide a multitude of clinical information, such as the pH of the aspirated fluid through a wide range of pH values. In various embodiments, when assembling pH indicator module 100, strip 120 is inserted inside barrel connector 110.

Returning to FIG. 1A, it depicts an assembled pH indicator module 100, according to some embodiments. In some embodiments, an assembled pH indicator module 100 is formed by assembling its parts in the order shown from bottom to top in FIG. 1B. In these embodiments, to assemble the parts, indicator strip 120 is folded, to form a “V” shape cross section shown in FIG. 1B. Strip 120 is then inserted into the barrel passageway 117; cap 130 is then fit over cap connector portion 119; and bushing 140 is then installed over cap barb 136. In such a configuration, a lower portion 124 of the folded strip includes a fold line that contacts or places on top of stop ledge element 118. The top two edges 126 of strip 120, on the other hand, contact the inner walls of barrel passageway 117. In various embodiments, indicator strip 120 fits and is maintained inside barrel passageway 117.

In the view shown in FIG. 1A, barrel connector 110 is rotated 90° compared to the view shown FIG. 1B. This view shows external surface 122 of strip 120. In various embodiments, a portion or all of surface 122 is visible through transparent barrel connector 110 on either side. Strip 120 is configured to indicate the pH of a gastric fluid via its color change. In some embodiments, such as the one shown in FIG. 1A, pH indicator module 100 also includes a label 128 for verifying the pH corresponding to the color change. Label 128 is a label that includes a color that matches a color of the strip 120 at a specific value or a range of values of pH. In various embodiments, label 128 is a colored piece of paper or similar material attached to pH indicator module 100 from inside or from outside. In some embodiments, label 128 is supplanted to pH indicator module 100 by ink pad printing at outside or inside.

FIG. 2B shows fluid aspiration system 200 in its operational mode. In FIG. 2B, a user pulls up plunger 214 to extract aspirated fluid 123 through connector passageway 111 into barrel passageway 117 of transparent barrel connector 110. As fluid enters barrel passageway 117, it contacts indicator strip 120, possibly causing strip 120 to change color. A user may compare the change of color in strip 120 with the color of label 128 and thus decide about the acidity of the aspirated fluid 123. In some embodiments, label 128 is colored to pantone red color 205U, which matches the red color change of a litmus paper strip 120 upon contacting an acid. Such a color change, thus, will confirm an acidic fluid, helping to confirm that the medical tube has been placed within the stomach.

In some embodiments, indicator strip 120 is configured to fit and remain inside barrel passageway 117. FIGS. 1C-1F depict strip 120 and its placement inside barrel passageway 117 according to some embodiments. In particular, FIG. 1C depicts a frontal view of a non-folded indicator strip 120 according to some embodiments. In some embodiment, indicator strip 120 is a rectangular blue litmus paper strip, with a width around ¼ inches and a length around 2 inches.

FIG. 1D depicts a side view of a folded indicator strip 120 according to some embodiments. In this folded configuration, a lower portion 124 of strip 120 produces an outward spring action at the top edges 126 of the folded strip 120.

In various embodiments, strip 120 is inserted into barrel passageway and is held in place without use of any adhesives. FIG. 1E shows strip 120 inserted into barrel passageway 117 according to some embodiments. In FIG. 1E, lower portion 124 rests in alignment on top of stop ledge element 118 and is thus held in place.

In some embodiments, top edges 126, on the other hand, are in contact with and press against the walls of barrel passageway 117 due to a spring force, shown by double arrow 129. In some embodiments, this contact produces a friction between top edges 126 and the walls, generating a mechanism for holding strip 120 in place.

In some embodiments, strip 120 is held in place by static friction forces between its various parts and the walls of barrel passageway 117. In some embodiments, strip 120 has a width 125 that is equal or slightly larger than a diameter of barrel passageway 117. Before being inserted into barrel passageway 120, in some embodiments strip 120 is folded and in some other embodiments strip 120 is not folded. Once inserted into barrel passageway 117, strip 120 will contact the inner walls of barrel passageway by its various parts, such as edges, 127. In some embodiments, because width 125 is slightly larger than the diameter of barrel passageway 117, edges 127 or other contacting surfaces exert a slight pressure against the inner wall of barrel passageway 117, providing for a friction force that holds strip 120 in place.

In some embodiments, one or more of the above mechanisms hold top edges 126 in place. The above mechanisms thus hold top edges 126 aligned with or close to a proximal end of barrel passageway 117, which abuts cap 130. The mechanisms further hold lower portion 124 aligned with or close to a distal end of barrel passageway 117, which abuts connector passageway 111.

FIG. 1F shows the set up of FIG. 1E when used to aspirate gastronomic fluid. In various embodiments, while strip lower portion 124 rests against or near stop ledge element 118, the set up shown in FIGS. 1E and 1F provides a fluid bypass area 121. In some embodiments, bypass area 121 has an inner circular opening of about 0.175 inches which provides an extensive flow path for the fluid to contact and circulate around strip 120. In some embodiments, strip 120 is suspended within barrel passageway 117 and cannot move forward or backwards within the passageway 117. Sufficient bypass area is provided around strip 120 to permit immersion of aspirated fluid to contact strip 120. In various embodiments, barrel passageway 117 is formed in various shapes, including cylindrical, oval, square, rectangular, or triangular.

Thus, when indicator module is used, fluid bypass area 121 permits aspirated fluid 123 (shown in FIG. 1F) to enter barrel passageway 117 and to contact strip 120. In various embodiments, aspirated fluid 123, when thus contacting strip 120, causes a change of color in strip 120 to indicate the pH of the aspirated fluid. For example, in some embodiments in which strip 120 is a blue litmus paper, an acidic stomach fluid causes strip 120 to turn red. Because barrel connector 110 is transparent, the color of strip 120 is visible from all or almost all angles around indicator module 100.

As seen in FIGS. 1D-1F, once strip 120 is folded to its V shape, it enables a user to view two strip surfaces 150 and 151 of strip 120. Moreover, barrel connector 110 includes transparent viewing window areas 152 and 153, through which the user can view strip surfaces 150 and 151. Such a set up facilitates viewing strip 120 at a wide range of light levels and viewing angles. In some embodiments, strip surfaces 150 and 151 are each dimensioned at around ¼ inches of width and around 1 inch of length. In some embodiments, each of barrel viewing windows 152 and 153 enables an approximately 180° viewing angle around barrel connector 110. Thus, together these windows enable a user to view strip 120 at any viewing angle around barrel connector 110 and independent of the angle at which strip 120 is inserted into barrel passageway 117. Strip 120 can thus be randomly and quickly inserted into barrel passageway 117 of barrel connector 110. This property speeds up production and lowers production labor cost for pH indicator module 100. Moreover, during clinical use, a user such as a clinician does not have to orient pH indicator module 100 into the stomach tube in any specific manner to be able to view the indicator. Instead, irrespective of the angle at which the tube enters the patient's body, the user can have a full 360° viewing angle of strip 120. Therefore, in various embodiments, pH indicator module 100 avoids the shortcomings of a pH indicator module that includes a single viewing window that is not visible from all angles. A single frontal side viewing window positioned within any type of aspirated fluid housing would be indirectly positioned away from any centrally aligned fluid flow path. Such indirectly positioned indicator strip design would be more complicated and expensive to produce.

In various embodiments, pH indicator module 100 also minimizes the required amount of aspirated fluid. In some embodiments, the required volume of aspirated stomach fluid 123 needed to cause a color change in strip 120 is less than 0.5 ml. pH indicator module 100 places indicator strip 120 in the direct fluid flow path of the aspirated fluid and thus requires aspiration of an amount of fluid that is enough to reach barrel connector 110. In some embodiments, pH indicator module 100 only requires aspirating around 0.1 ml of fluid for the fluid to contact indicator strip 120. Such a mechanism avoids shortcomings of a pH indicator system in which a pH indicator is placed in a separate chamber away from the main flow path. This separate chamber design requires aspirating an additional amount of fluid to reach the separate chamber. pH indicator module 100 is thus usable in situations in which the separate chamber design will not be convenient or usable; e.g., situations where only a small amount of fluid can be aspirated. These cases include using pH indicator module 100 with small bore neonate and pediatric stomach tubes of 3.5fr and 5fr., which usually deliver gastric aspirate of less than 1 ml.

In some embodiments, connector passageway 111 has a flow path of about 0.090 inches (2.3 mm) in diameter and a length of 1¼ inches. With these dimensions, connector passageway 111 provides an adequate flow path with a very low volume of fluid of about 0.2 ml. The indicator strip 120 is positioned in barrel passageway 117 right after connector passageway 111 and in the direct path of the flow of the aspirated fluid. Therefore, the amount of fluid that a user should aspirate for the fluid to reach strip 120 is only slightly more than the amount of fluid needed to fill connector passageway 111. As a result, in some embodiments the total volume of stomach fluid 123 that a user should aspirate for the fluid to reach contact strip 120 is about 0.4 ml. In some embodiments, this property enables the pH indicator module to be used with smaller French sized nasoenteric tubes such as models 5, 6, and 8, which only yield an aspirated volume of 1 ml to 3 ml.

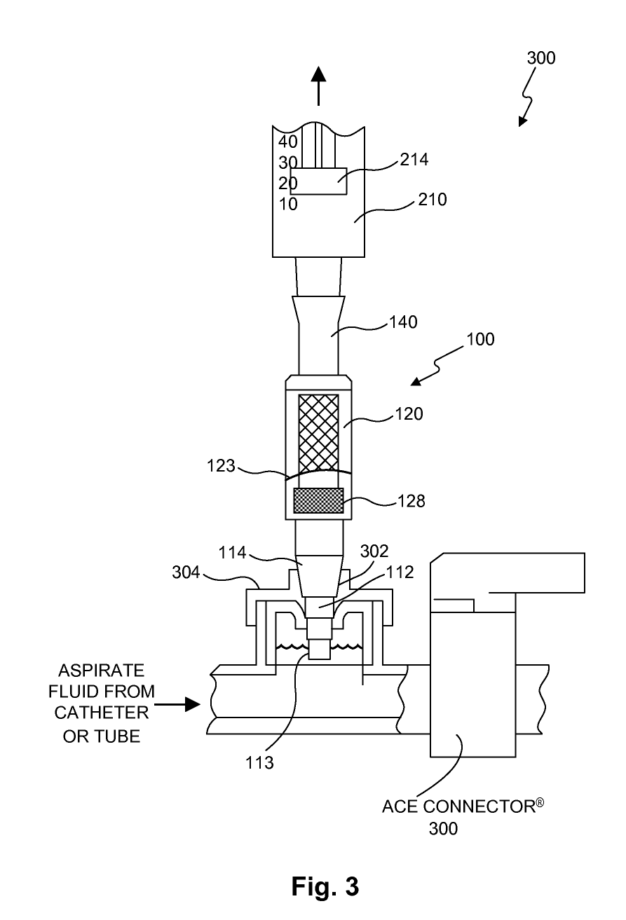

FIG. 3 shows a system 300 in which pH indictor module 100 attaches to an ACE Connector® 300, according to an embodiment. In FIG. 3, tube connector portion 112 and enlarged tapered portion 114 form a secure wedge lock leakproof seal 302 with syringe port cap 304. This combination permits nozzle tip 113 to aspirate fluid 123 via syringe 210 for the fluid to reach indicator strip 120.

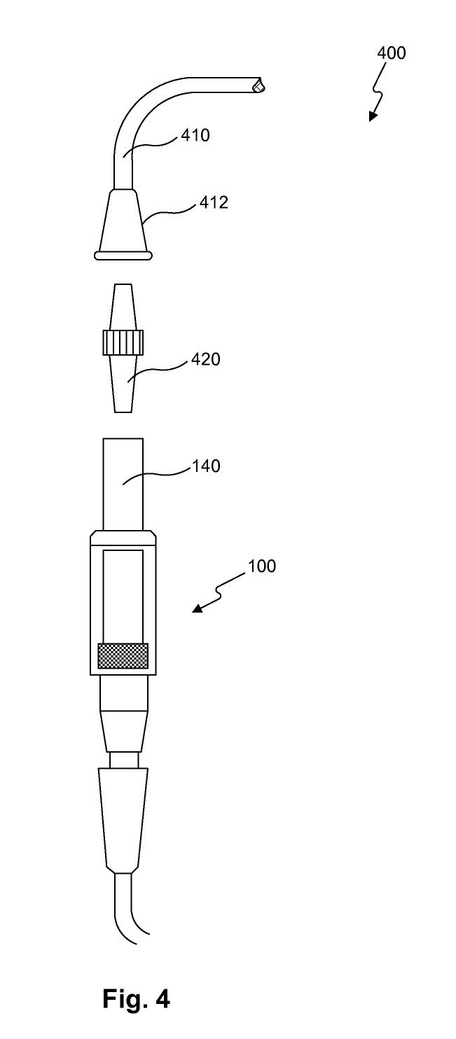

FIG. 4 shows a system 400 in which a pH indictor module is used with an aspiration suction source other than a syringe, in accordance with some embodiments. In system 400, pH indicator module 100 attaches to an aspiration suction source tubing line 410. In some embodiments, tubing line 410 includes, at one end, a connector capping 412. In some embodiments, as shown in FIG. 4, pH indicator attaches to tubing line 410 through connector 420. In particular, connector 420 fits, at its two ends, inside connector capping 412 and busing 140. In some embodiments, connector 420 is a SIMS or 5 in 1 connector. In some embodiment, aspiration suction source tubing line 410 connects at another end to a bedside wall suction.

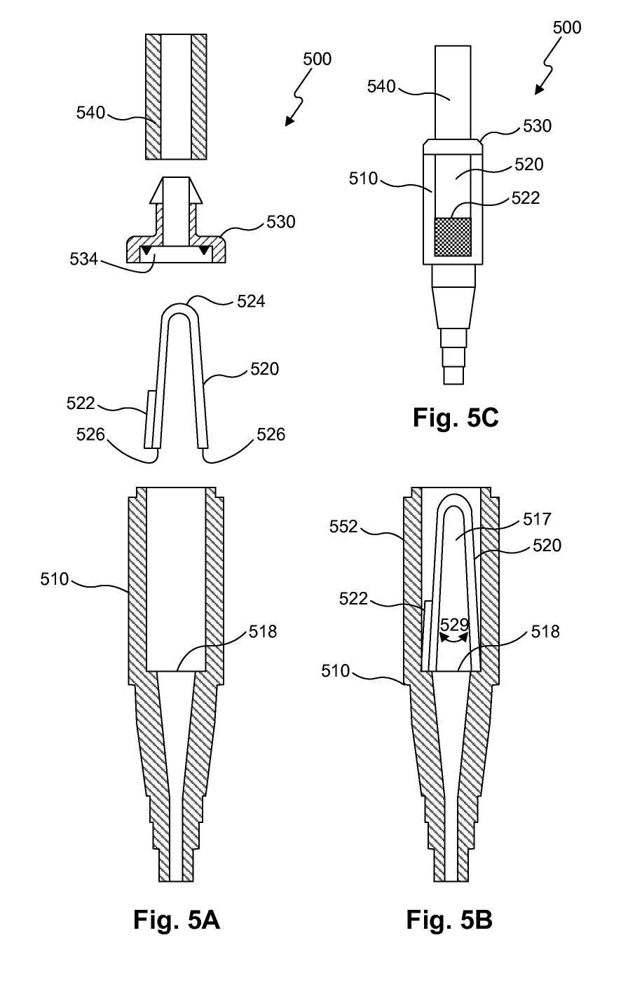

FIGS. 5A-5C depict a pH indicator module 500 according to another embodiment. As seen in FIG. 5A, pH indicator module 500 includes barrel connector 510, indicator strip 520, cap 530, and bushing 540. In various embodiments, indicator strip 520 is a broad spectrum flexible plastic pH indicator strip, which is attached to a color change panel 522. Color change panel 522, when contacting aspirated fluid, shows a spectrum of three or more colors based on the pH of the fluid. In one embodiment, color change panel 522 is a strip manufactured by Micro Essential Laboratories catalog no. 9200. In that embodiment, color change panel 522, when exposed to gastric fluid, will indicate a clear visible color change in on-half increments of pH value, from 0.0 (highly acidic) to 6.0 (very alkaline).

FIG. 5B shows a configuration for inserting indicator strip 520 in barrel connector 510. In particular, as seen in FIGS. 5A and 5B, indicator strip 520 is folded in a V shape form and is inserted in barrel passageway 517 of barrel connector 510. In the embodiment shown in FIGS. 5A-5C, the folded strip 520 is inserted in a converted V orientation. In this manner, the two edges 526 of folded strip 520 rest in alignment or on top of stop ledge element 518 and are thus held in place. In some embodiments, the fold portion 524 of folded strip 520, on the other hand, is held in place by resting against ring 534 or some sections of the wall of cap 530. In some embodiments, edges 526 push against the internal walls of barrel passageway 517 due to an outward spring force 529 of folded strip 520. As shown in FIGS. 5A-5C, in this configuration, color change panel 522 is visible through transparent viewing window 552 of barrel connector 510. In some embodiments, barrel connector 510 is transparent and viewing window 552 provides an angle of around 180° for viewing color change panel 522.

In some embodiments, not one, but two color change panels 522 are attached to indicator strip 520. In this manner, when strip 520 is folded and inserted into barrel passageway 517, the two color change panels 522 can be seen from either side of pH indicator module 500, providing for a viewing angle of about 36⁰°. In some embodiments, folded strip 520 is inserted into barrel passageway 517 not in the inverted V orientation of FIG. 5B, but in an upright orientation similar to that shown for strip 120 in FIGS. 1D-1F.

FIG. 5C shows a side view of fully assembled pH indicator module 500 in accordance to some embodiments. As shown, when strip indicator strip 520 is exposed to aspirated gastric fluid strip 520 and color change panel 522 will be fully visible through transparent barrel connector 510.

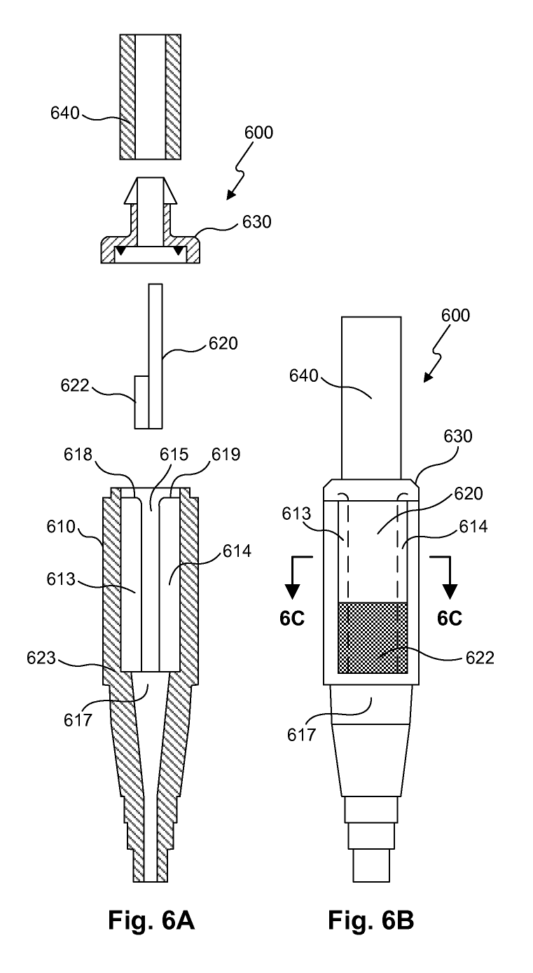

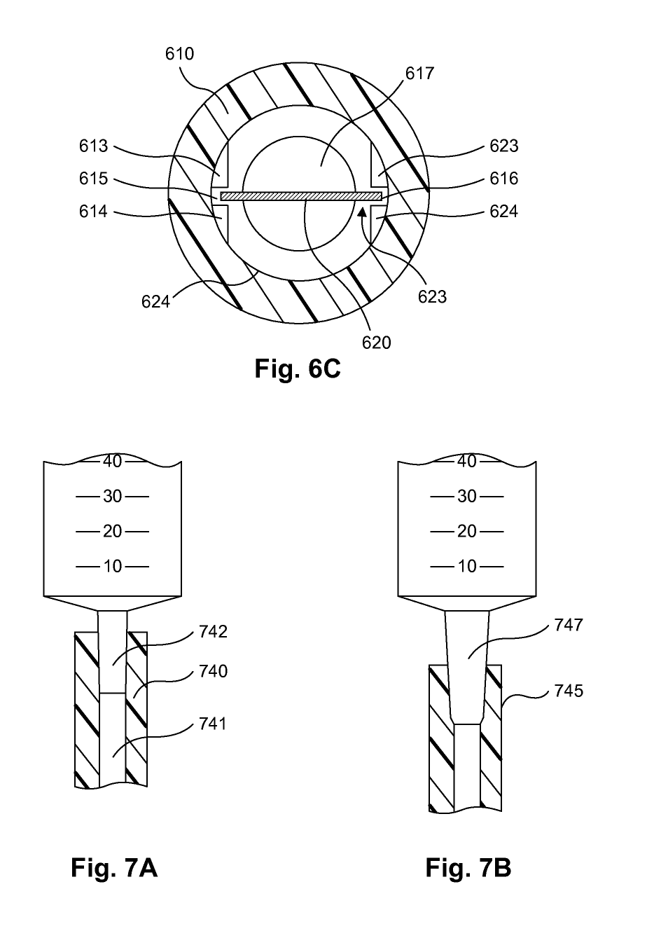

FIGS. 6A-6C depict a pH indicator module 600 according to another embodiment. In particular, FIG. 6A shows an exploded view of pH indicator module 600 while FIG. 6B shows a side view of an assembled pH indicator module 600. As seen in FIGS. 6A and 6B, pH indicator module 600 includes barrel connector 610, indicator strip 620, cap 630, and bushing 640. In some embodiments, indicator strip 620 is attached to a color change panel 622. In some embodiments, indicator strip 620 is inserted into barrel connector 610 in the form shown in FIG. 6A and without being folded. In some embodiment, non-folded indicator strip 620 is inserted into a slot groove formed in barrel connector 610.

Barrel connector 610 includes molded retention bars 613 and 614, which together form a central molded slot groove 615. Retention bars 613 and 614 respectively have tapered or rounded entrance guide edges 618 and 619. Edges 618 and 619 facilitate insertion of indicator strip 620 into slot groove 615. Barrel connector 610 also forms barrel passageway 617 through which aspirated fluid flows.

FIG. 6C shows an enlarged cross sectional view through barrel connector 610 taken along lines 6C-6C shown in FIG. 6B. FIG. 6C depicts strip 620 inserted into slot grooves 615 and 616 and held in place by molded in retention bars 613, 614, 623, and 624. Transition area 623 retains strip 620 such that aspirated gastric fluid flowing upward through central barrel passageway 617 contacts strip 620.

In various embodiments, strip 620 is made from a high density plastic polymer that is flexible and also firm. As such, strip 620 can be inserted into slot grooves 615 and 616, and held in place with no need for adhesives, glues, or other strip retention components. Such a mechanism reduces the cost of material and labor in manufacturing pH indicator module 600. In some embodiments, strip 620 is about 0.010 inches (0.24 mm) in thickness, and can thus be easily inserted into slightly wider slot grooves 615 and 616.

In some embodiments, inner wall 624 of barrel connector 610 is dimensioned slightly below the nominal width of strip 620. For example, in some embodiments, this nominal width is typically about 0.265 inches and the inner wall 624 is dimensioned to about 0.255 inch J.D. In some embodiments, the oversized strip width generates a slight interference fit, eliminating the need for slot grooves 615 or 616.

FIG. 7A depicts a flexible PVC bushing 740 engaging an oral centric syringe tip 742 according to some embodiments. Similarly, FIG. 7B depicts a flexible bushing 745 engaging a larger tip 60 cc catheter tip syringe 747 according to some embodiments. In some embodiments, flexible bushing 740 is either extruded or molded from flexible PVC or silicone with an interior central flow path 741 of about 0.215 (5.46 mm). Further, in some embodiments, flexible bushing 745 is molded from flexible polymer of about 60 shore A durometer.

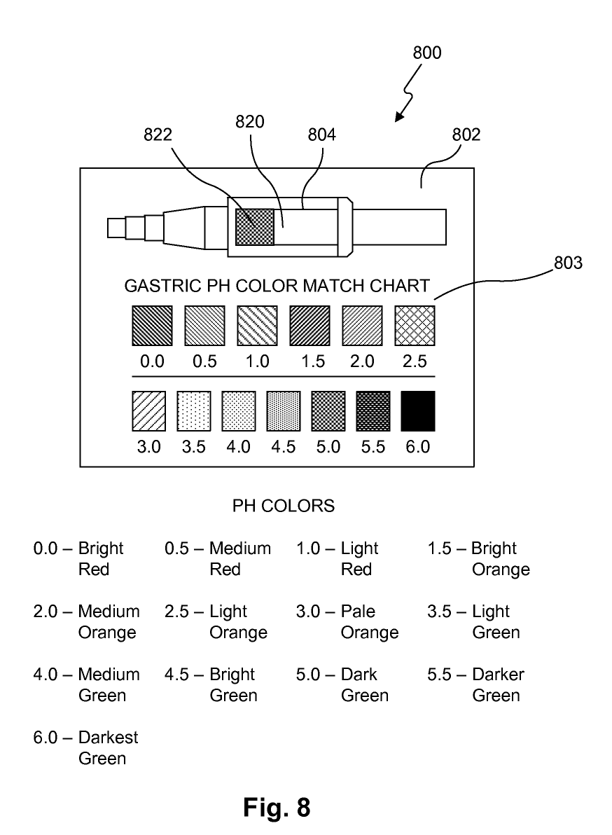

FIG. 8 depicts a pH indicator package 800 according to some embodiments. Package 800 includes a pouch 802 and a pH indicator module 804. pH indicator module 804 includes a strip 820 to which a color change panel 822 is attached. Pouch 802 includes a pre-printed pH color match chart 803 printed as part of the pouch. In one embodiment, as shown in FIG. 8, chart 803 shows 13 distinct colors corresponding to pH values ranging from 0.0 (highly acidic) to 6.0 (very alkaline), in 0.5 increments. When pH indicator module 804 is exposed to aspirated gastric fluid, color change panel 822 changes color to the respective color corresponding the pH of gastric fluid in one-half increments. The color of color change panel 822 can be compared with the colors in chart 803 to determine the pH.

It will be apparent to those skilled in the art that additional various modifications and variations can be made consistent with the present disclosure. Other embodiments consistent with the tube holder will be apparent to those skilled in the art from consideration of the specification and practice of the disclosure. It is intended that the specification and examples be considered as exemplary only, with a true scope and spirit of the disclosure being indicated by the following claims.

1. A pH indicator module configured to attach on a proximal module end to a suction mechanism and on a distal module end to a proximal tube end of an aspiration tube, and configured to be used during aspirating an aspirated fluid drawn by the suction mechanism from a location of a distal tube end of the aspiration tube, the pH indicator module comprising:
a barrel connector configured to attach to the aspiration tube, wherein the barrel connector forms an inner passageway and wherein the passageway is located in a direct flow path of the aspirated fluid when the pH indicator module is used, and wherein the barrel connector is configured to receive a pH indicator material placed in the passageway, wherein the pH indicator material is configured to come into contact with a direct flow of the aspirated fluid drawn into the passageway to determine a pH value of the aspirated fluid; and
a cap portion configured to attach on a proximal cap end to the suction mechanism and to attach on a distal cap end to the barrel connector.

2. The pH indicator module of claim 1, wherein the pH indicator material is a pH indicating strip of material.

3. The pH indicator module of claim 2, wherein the pH indicator material is shaped in a rectangle and is folded in a V shape cross section before being placed in the passageway.

4. The pH indicator module of claim 2, wherein the pH indicating strip is fixed inside the passageway, at least in part, by contacting an inner surface of the passageway.

5. The pH indicator module of claim 1, wherein the barrel connector is made of a transparent material, and wherein the pH indicator material is visible from a viewing angle greater than or equal to 180° around the pH indicator module.

6. The pH indicator module of claim 1, wherein the barrel connector is sized such that during the aspirating less than 0.5 ml of the aspirated fluid is required to enter the pH indicator module for the aspirated fluid to come into contact with the pH indicator material.

7. The pH indicator module of claim wherein a slot is formed in the passageway and wherein the pH indicating strip of material is inserted in the slot and held in place, at least in part, by contacting internal walls of the slot.

8. The pH indicator module of claim 1, further comprising a label including a label color that matches an indicator color of the pH indicator material when during the aspirating the pH indicator material comes into contact with the aspirated fluid and when the aspirated fluid has a pH value in a specific range.

9. The pH indicator module of claim 1, wherein the cap portion includes at the proximal cap end a bushing section configured to attach to the suction mechanism and further includes at the distal cap end a cap section configured to attach to the barrel connector.

10. The pH indicator module of claim 1, wherein the pH indicator material is configured to determine that the pH value falls into one of more than two pH value ranges.

11. The PH indicator module of claim 1, wherein the proximal cap end is configured to be connected to a syringe.

12. The PH indicator module of claim 1, wherein the pH value is used to determine whether a distal tube end of a gastric tube has been properly placed within a patient's stomach.

13. The pH indicator module of claim 1, wherein the pH indicator material is placed in a center of the passageway.

14. The pH indicator module of claim 1, wherein the passageway is cylindrical.

15. The pH indicator module of claim 1 further comprising the pH indicator material, wherein the pH indicator material is inserted and held in place in the passageway, such that the pH indicator material is configured to come into contact with the direct flow of the aspirated fluid drawn into the passageway.

16. The PH indicator module of claim 1, wherein the proximal cap end includes a luer fitting configured to be connected to a luer syringe.

17. A method of using a pH indicator module to measure a pH value of an aspirated fluid, wherein the pH indicator module is configured to attach on a proximal module end to a suction mechanism and on a distal module end to a proximal tube end of an aspiration tube, and to be used during aspirating the aspirated fluid drawn by the suction mechanism from a location of a distal tube end of the aspiration tube, the method comprising:
providing a barrel connector included in the pH indicator module, the barrel connector configured to attach to the aspiration tube, wherein the barrel connector forms an inner passageway, and wherein the passageway is located in a direct flow path of the aspirated fluid when the pH indicator module is used;
providing a pH indicator material inserted in the passageway, wherein the pH indicator material is configured to come into contact with a direct flow of the aspirated fluid drawn into the passageway to determine a pH value of the aspirated fluid; and
providing a cap portion included in the pH indicator module, wherein the cap portion is configured to attach to the suction mechanism on a proximal cap end and to attach to a proximal barrel end of the barrel connector on a distal cap end;
attaching a distal barrel end of the barrel connector to the aspiration tube;
drawing a fluid into the passageway using the suction mechanism; and
observing a color of the pH indicator material after contacting the fluid.

18. The method of using a pH indicator module of claim 17, wherein the pH indicator material is a pH indicating strip of material, and the pH value is used to determine whether a distal tube end of a gastric tube has been properly placed within a patient's stomach.

19. The method of using a pH indicator module of claim 18, wherein the pH indicator material is shaped in a rectangle and is folded in a V shape cross section before being placed in a center of the passageway.

20. The method of using a pH indicator module of claim 18, wherein the pH indicating strip is fixed inside the passageway, at least in part, by contacting an inner surface of the passageway.

21. The method of using a pH indicator module of claim 17, wherein the barrel connector is made of a transparent material, and wherein the pH indicator material is visible from a viewing angle greater than or equal to 180° around the pH indicator module.

22. The method of using a pH indicator module of claim 18, wherein a slot is formed in the passageway and wherein the pH indicating strip of material is inserted in the slot and held in place, at least in part, by contacting internal walls of the slot.

23. The method of using a pH indicator module of claim 17, further comprising providing a label including a label color that matches an indicator color of the pH indicator material when during the aspirating the pH indicator material comes into contact with the aspirated fluid and when the aspirated fluid has a pH value in a specific range.

24. The method of using a pH indicator module of claim 17, wherein the cap portion includes at the proximal cap end a bushing section configured to attach to the suction mechanism and further includes at the distal cap end a cap section configured to attach to the barrel connector.

25. A pH indicator module comprising:
a proximal end configured to attach to a suction mechanism;
a distal end configured to attach to a an aspiration tube;
a barrel connector including a wall that forms both an outer surface of said barrel connector and an inner passageway, wherein during use the passageway is located in a direct flow path of the aspirated fluid; and
a pH indicator material placed in the inner passageway, wherein during use the pH indicator material is configured to come into contact with a direct flow of the aspirated fluid drawn into the passageway to determine a pH value of the aspirated fluid.
